(19) United States
(12) Patent Application Publication
(10) Pub. No.: US 2006/0276437 A1
(43) Pub. Date: Dec. 7, 2006

(54) Process for producing optically active dihydropyridinephosphonic ester

(76) Inventors: Yasutaka Takada, Funabashi-shi (JP); Hiroo Matsumoto, Funabashi-shi (JP)

Correspondence Address: OLIFF & BERRIDGE, PLC, P.O. BOX 19928, ALEXANDRIA, VA, 22320, US

(21) Appl. No.: 10/573,972

(22) PCT Filed: Aug. 12, 2004
(86) PCT No.: PCT/JP04/11607

§ 371 Date: Mar. 30, 2006

(30) Foreign Application Priority Data
Oct. 1, 2003 (JP) 2003-342761

(51) Int. Cl. A61K 31/675; C07F 9/58

(52) U.S. Cl. 514/89; 546/21

(57) ABSTRACT

There is provided a process for efficiently producing an optically active substance of efonidipine that is useful as an antihypertensive agent or a therapeutic agent for angina pectoris.
A process for producing an optically active substance of compound of formula (1) 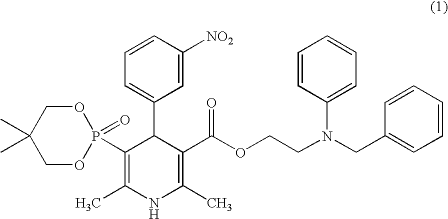 characterized by comprising: dissolving a racemate of the compound of formula (1) in a solvent to prepare a supersaturated solution; and adding crystal of either one optically active substance of the compound of formula (1) as a seed crystal in the supersaturated solution to allow crystal of the one optically active substance added as the seed crystal to separate out; or dissolving a mixture of the compound of formula (1) in which either one optically active substance thereof is present in excess in a solvent to prepare a supersaturated solution; and adding crystal of the one optically active substance present in excess as a seed crystal in the supersaturated solution to allow crystal of the one optically active substance present in excess to separate out.

### TECHNICAL FIELD

The present invention relates to a process for efficiently producing an optically active substance of efonidipine (2-[Benzyl(phenyl)amino]ethyl 5-(5,5-dimethyl-2-oxo-1,3,2-dioxaphosphorinan-2-yl)-1,4-dihydro-2,6-dimethyl-4-(3-nitrophenyl)-3-pyridinecarboxylate) that is useful as an antihypertensive agent or a therapeutic agent for angina pectoris. Specifically, the present invention relates to a process for efficiently producing an optically active substance of efonidipine by preferential crystallization.

### BACKGROUND ART

Efonidipine (2-[Benzyl(phenyl)amino]ethyl 5-(5,5-dimethyl-2-oxo-1,3,2-dioxaphosphorinan-2-yl)-1,4-dihydro-2,6-dimethyl-4-(3-nitrophenyl)-3-pyridinecarboxylate) is placed on the market as an antihypertensive agent or a therapeutic agent for angina pectoris, and it has been already clarified that the antihypertensive activity is attributed to one optically active substance (S(+) form) (see, for example, Patent Document 1 and Non-patent Document 1).

As a process for producing an optically active substance of efonidipine, the following three processes have been already reported:

(1) Diastereomer resolution through (2S)-2-methoxy-2-phenylethyl(4S)-5-(5,5-dimethyl-2-oxo-1,3,2-dioxaphosphorinan-2-yl)-1,4-dihydro-2,6-dimethyl-4-(3-nitrophenyl)-3-pyridine-carboxylate, shown in FIG. 1 below (see, for example, Patent Document 2 and Non-patent Document 2)

[image]

(2) Production method by utilizing enzyme shown in FIG. 2 below (see, for example, Non-patent Documents 3 and 4)

[image]

(3) Method by optical resolution with optical isomer-separation column (see, for example, Non-patent Document 5).

Patent Document 1: JP-A-63-233992 (1988)
Patent Document 2: JP-A-2-11592 (1990)
Non-patent Document 1: Y Masuda, T. Sakai, M. Sakasita, M. Takeguti, T. Takahashi, C. Arakawa, M. Hibi, S. Tanaka, K. Shigenobu, and Y. Kasuya, Jpn. J. Pharmacol., 48, p. 266 (1988)
Non-patent Document 2: R. Sakoda, H. Matsumoto, K. Seto, Chem. Pharm. Bull., 40(9), p. 2377 (1992)
Non-patent Document 3: T. Kato, M. Teshima, H. Ebiike, and K. Achiwa: Chem. Pharm. Bull., 44, p. 1132 (1996)
Non-patent Document 4: H. Ebiike, Y Yamazaki, and K. Achiwa: Chem. Pharm. Bull., 43, p. 1251 (1995)
Non-patent Document 5: New Development of Process Chemistry (2003), p. 253, CMC Publishing Co., Ltd., Edit.: The Japanese Society of Process Chemistry Disclosure Of Invention
   Problem to be Solved by the Invention

The diastereomer resolution of (1) is inappropriate as an industrial production process as optically active alcohol used is expensive, a long time is required for steps, the yield of transesterification is low, and the introduction and removal of protective groups are required.

The production method (asymmetric synthesis) by use of enzyme of (2) is good in the yield of the step for obtaining an optically active dihydropyridine-dicarboxylate, but many steps are required for inducing the optically active substance of efonidipine, and the method has disadvantages that the yield of transesterification is low, materials are expensive and the like from the standpoint of industrial production methods. The optical resolution by use of optical isomer-separation column of (3) is a technique that is industrially applicable, but it has problems that an expensive packing is used, a large amount of solvent is used, and a special equipment (simulated moving bed system) is required.

The problem to be solved by the present invention is to establish a process for industrially producing an optically active substance of efonidipine that is economically excellent.

Means for Solving the Problem

The present inventors eagerly investigated as to processes for efficiently producing an optically active substance of efonidipine. As a result of it, they noticed that efonidipine is a racemic mixture, found that the optically active substance can be easily produced bay carrying out preferential crystallization, and completed the present invention.

That is, the present invention relates to

1. A process for producing an optically active substance of compound of formula (1) 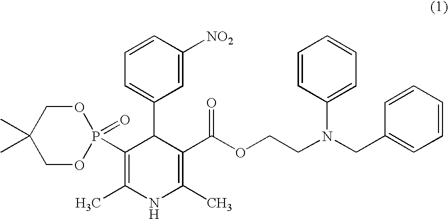
characterized by comprising:
dissolving a racemate of the compound of formula (1) in a solvent to prepare a supersaturated solution; and
adding crystal of either one optically active substance of the compound of formula (1) as a seed crystal in the supersaturated solution to allow crystal of the one optically active substance added as the seed crystal to separate out; or
dissolving a mixture of the compound of formula (1) in which either one optically active substance thereof is present in excess in a solvent to prepare a supersaturated solution; and
adding crystal of the one optically active substance present in excess as a seed crystal in the supersaturated solution to allow crystal of the one optically active substance present in excess to separate out;

2. The process for producing an optically active substance as set forth in 1., wherein the solvent is alcohols or esters;

3. A process for purifying an optically active substance of compound of formula (1) 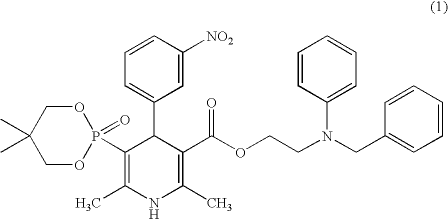
characterized by comprising:
recrystallizing a mixture of the compound of formula (1) in which either one optically active substance thereof is present in excess, or
allowing crystal to separate out from a solution of a mixture of the compound of formula (1) in which either one optically active substance thereof is present in excess, as a result of it, an optically active substance present in excess in a mother liquor from which the crystal is removed is the other optically active substance that is not one of the crystal;

4. The process for purifying an optically active substance as set forth in 3., wherein the solvent is alcohols or esters;

5. A process for producing an optically active substance of compound of formula (1) 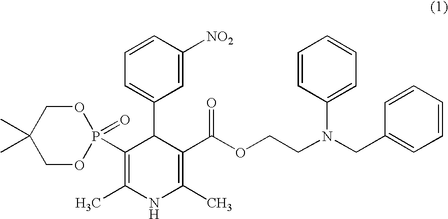
characterized by comprising:
dissolving a mixture of the compound of formula (1) in which either one optically active substance thereof is present in excess in an aromatic hydrocarbon of formula (2) 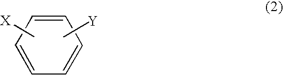
wherein X and Y are identical with or different from each other, hydrogen atom, C₁₋₃alkyl group, halogen atom or C₁₋₃alkoxy group, to prepare a supersaturated solution;
crystallizing a solvate of a racemate of the compound of formula (1) and the aromatic hydrocarbon from the supersaturated solution;
removing resulting crystal, and then
obtaining in a high purity the one optically active substance present in excess of the compound of formula (1).

The preferential crystallization can provide an optically active substance in a simple mode equivalent to recrystallization process of racemates if only the seed crystal is present, and is an extremely useful process from the industrial and economical standpoint.

Best Mode for Carrying Out the Invention

Hereinafter, the present invention is described in further detail.

In the meantime, “n” means normal, “i” means iso, and “c” means cyclo in this specification.

Each substituent described in this specification is explained.

Halogen atom includes fluorine atom, chlorine atom, bromine atom and iodine atom. C₁₋₃alkyl group may be straight-chain, branched or cyclic, and includes for example methyl group, ethyl group, n-propyl group, i-propyl group and c-propyl group. C₁₋₃alkoxy group may be straight-chain, branched or cyclic, and includes for example methoxy group, ethoxy group, n-propoxy group, i-propoxy group and c-propoxy group.

The racemate of the compound of formula (1) used in the present invention can be easily produced according to the production method described in JP-A-63-233992 (1988). The crystal of the optically active substance of the compound of formula (1) used as a seed crystal in the present invention can be produced by use of the production method described in JP-A-2-11592 (1990), or an optical resolution with optical isomer-separation column, or the like.

The production of the optically active substance of the compound of formula (1) according to the present invention is achieved by adding crystal of either one optically active substance of the compound of formula (1) as a seed crystal in a supersaturated solution of racemate of the compound of formula (1), and allowing crystal to separate out, or by adding crystal of either one optically active substance of the compound of formula (1) as a seed crystal in a supersaturated solution in which the either one optically active substance is present in excess, and allowing crystal to separate out.

The supersaturated solution can be prepared according to any conventional method such as a method comprising dissolving with heat a racemate of the compound of the formula (1) or a mixture in which either one optically active substance is present in excess in a suitable solvent, and then cooling the resulting solution, or a method comprising concentrating the solution, or a method comprising adding a solvent that lowers solubility in the solution, or the like.

When the optically active substance of the compound of the formula (1) is produced by use of a mixture in which either one optically active substance is present in excess, the crystal of the optically active substance as a seed crystal is not necessarily added externally, and an optical resolution proceeds also by natural crystallization.

The solvents used in the process for producing the optically active substance of the compound of the formula (1) are not specifically limited so long as the compound of the formula (1) shows a suitable solubility, and includes for example alcohols such as methyl alcohol, ethyl alcohol, 1-propanol and 2-propanol, or the like, esters such as ethyl acetate or the like, ketones such as methyl isobutyl ketone and acetone, or the like, aliphatic hydrocarbons such as n-hexane, n-heptane and cyclohexane, or the like, ethers such as isopropyl ether and tetrahydrofuran, or the like; halogen-containing solvents such as dichloromethane and chloroform, or the like, aprotic polar solvents such as dimethylformamide and dimethylsulfoxide, or the like, and mixed solvents in which two or more of the above-mentioned solvents are contained in an arbitrary proportion. Preferable solvents include for example alcohols and esters, and particularly preferable solvents include for example alcohols. Preferable concrete solvents include for example methyl alcohol, ethyl alcohol, 2-propanol and ethyl acetate, more preferable solvents include for example methyl alcohol and ethyl alcohol, and further preferably it includes methyl alcohol.

The amount of the solvent used is not specifically limited so long as it is an amount range in which a racemate of the compound of formula (1) or a mixture in which either one optically active substance is present in excess becomes supersaturation. However, it is preferable to carry out in a suitable range because in case where the used amount is too much, the resulting crystal is lowered in amount, and in case where the used amount is too small, there is fear that the optical purity of the resulting crystal is lowered.

As the solubility of the compound of formula (1) differs from each other depending on the kind of the solvents, suitable used amount of the solvents cannot be generally determined. However, for example, when alcohols or esters are used as solvent, the used amount of the solvent is preferably 10 to 50 times by mass, more preferably 15 to 30 times by mass based on the used amount of the racemate of the compound of formula (1) or the mixture in which either one optically active substance is present in excess.

The added amount and grain size of the crystal of the optically active substance of the compound of formula (1) that is used as seed crystal are not specifically limited. However, it is used in an amount of 0.1 to 20 mass %, preferably 0.5 to 5 mass % based on the racemate of the compound of formula (1) or the mixture in which either one optically active substance is present in excess, in a form of ground powder.

The temperature on preparing the supersaturated solution by dissolution with heat is not specifically limited and may range from room temperature to a boiling point of the solvent. However, it is required to be controlled so that a stably supersaturated solution could be obtained depending on the solubility of the compound of formula (1) in the used solvent. For example, in case where alcohols or esters are used as a solvent, the temperature ranging from 50° C. to the boiling point of the solvent is often used as a temperature on preparing the supersaturated solution by dissolution with heat.

The temperature on adding the crystal of the optically active substance of the compound of formula (1) as a seed crystal is not generally determined as it varies depending on the kind and used amount of the solvent used. However, for example, in case where alcohols or esters are used as a solvent, the temperature ranging from 40° C. to 60° C. is preferably used as a temperature on adding the crystal of the optically active substance.

The temperature on allowing crystal to separate out is not generally determined as it varies depending on the kind and used amount of the solvent used. However, for example, in case where alcohols or esters are used as a solvent, the temperature ranging from 20° C. to 40° C. is preferably used as a temperature on allowing crystal to separate out.

The time required for allowing crystal to separate out is not specifically limited so long as optical purity can be secured to some extent, but about 30 minutes to about hours are sufficient therefor. In addition, the methods for allowing crystal to separate out include a method by permitting to stand or a method by carrying out with stirring, etc., but it is preferable to be carried out with stirring.

In industrial production of the optically active substance of the compound of formula (1), the following several methods that are known as preferential crystallization can be used: a batch method in which one optically active substance and the other optically active substance are resolved alternately, a continuous method in which a supersaturated solution is continuously flowed through a column in which a seed crystal is present, or a method in which one optically active substance and the other optically active substance are immersed as seed crystals in places separated by a distance and each seed crystal is grown, or the like.

When the optically active substance obtained as mentioned above is insufficient in optical purity, the optical purity can be further increased by recrystallization or the like. In this case, as an optically active substance present in excess in the mother liquor after recrystallization is different from the crystallized optically active substance, it is said to be an extremely efficient method from the standpoint of the efficiency of purification.

Similarly efficient purification can be carried out also by recrystallizing crystal of a mixture in which either one optically active substance is present in excess, or allowing crystal to separate out from a mixture in which either one optically active substance is present in excess (that is, an optically active substance that becomes excess in the mother liquor is different from the crystallized optically active substance).

The purification method is a means particularly effective for purifying a mixture of the compound of formula (1) having a high optical purity, for example for purifying a mixture having an optical purity of 70% ee or more, preferably 80% ee or more.

The solvents used in the purification method are not specifically limited so long as the compound of the formula (1) shows a suitable solubility, and includes for example alcohols such as methyl alcohol, ethyl alcohol, 1-propanol and 2-propanol, or the like, esters such as ethyl acetate or the like, ketones such as methyl isobutyl ketone and acetone, or the like, aliphatic hydrocarbons such as n-hexane, n-heptane and cyclohexane, or the like, ethers such as isopropyl ether and tetrahydrofuran, or the like, halogen-containing solvents such as dichloromethane and chloroform, or the like, aprotic polar solvents such as dimethylformamide and dimethylsulfoxide, or the like, and mixed solvents in which two or more of the above-mentioned solvents are contained in an arbitrary proportion. Preferable solvents include for example alcohols and esters, and particularly preferable solvents include for example alcohols. Preferable concrete solvents include for example methyl alcohol, ethyl alcohol, 2-propanol and ethyl acetate, more preferable solvents include for example methyl alcohol and ethyl alcohol, and further preferably it includes methyl alcohol.

In addition, as the condition of crystallization, the condition similar to that in the process for producing optically active substance of the compound of formula (1) except that seed crystal is not used can be used.

When the compound of formula (1) is crystallized from aromatic hydrocarbons, the crystal is obtained as a solvate of racemate, but the optically active substance of the compound of formula (1) does not form any solvate. Further, the resulting crystal of solvate of racemate of compound of formula (1) has an extremely low solubility in the used aromatic hydrocarbons, and therefore either one optically active substance of the compound of formula (1) can be produced in a high purity from the mother liquor from which the crystal is removed.

The aromatic hydrocarbons that can form the solvate of racemate of compound of formula (1) include aromatic hydrocarbons of formula (2) 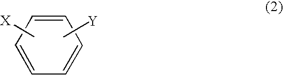
wherein X and Y are identical with or different from each other, hydrogen atom, C₁₋₃alkyl group, halogen atom or C₁₋₃alkoxy group. Preferable aromatic hydrocarbons include toluene and xylene.

The amount of the solvent used is not specifically limited so long as it is an amount range so that a mixture in which either one optically active substance of the compound of formula (1) is present in excess becomes supersaturation. However, it is preferably 10 to 50 times by mass based on the used amount of the mixture in which either one optically active substance of the compound of formula (1) is present in excess.

When the crystal of solvate of racemate of compound of formula (1) is separated out, any seed crystal is not generally required. However, the crystal of solvate of racemate of compound of formula (1) may be added as seed crystal. In case where the crystal of solvate of racemate of compound of formula (1) is added as seed crystal, the added amount and grain size are not specifically limited. However, it is used in an amount of 0.1 to 20 mass %, preferably 0.5 to 5 mass % based on the racemate of the compound of formula (1) or the mixture in which either one optically active substance is present in excess, in a form of ground powder.

The temperature on preparing the supersaturated solution by dissolution with heat is not specifically limited and may range from room temperature to a boiling point of the solvent. However, it is required to be controlled so that a stably supersaturated solution could be obtained depending on the solubility of the compound of formula (1) in the used solvent. The temperature ranging from 50° C. to a boiling point of the solvent is often used as a temperature on preparing the supersaturated solution by dissolution with heat depending on the kind and amount of the solvent used. The temperature on adding the crystal of solvate of racemate of the compound of formula (1) as a seed crystal is preferably 40° C. to 60° C. The temperature on allowing crystal to separate out is preferably 0° C. to 40° C.

The time required for separating out the crystal is not specifically limited so long as optical purity can be secured to some extent, but about 30 minutes to about 5 hours are sufficient therefor. In addition, the methods for allowing crystal to separate out include a method by permitting to stand or a method by carrying out with stirring, but it is preferable to be carried out with stirring.

### EXAMPLES

Hereinafter, the present invention will be concretely described based on examples to which the present invention is not limited.

In the meantime, an optically active substance of the compound of formula (1) used in the examples was prepared according to Example 25 of JP-A-63-233992 (1988) as follows: a racemate of compound of formula (1) was synthesized and then the optionally active substance was collected with HPLC through an optical isomer-separation column.

HPLC collection condition

Column: CHIRALCELOC (manufactured by Daicel Chemical Industries, Ltd.);

Column Size: 20 cmφ×50 cmφ

Eluent: Methanol

Column Temperature: Room temperature

Flow Rate: 760 mL/min.

In addition, the optical purity of the optically active substance of the compound of formula (1) obtained in each example was analyzed in a condition of eluent: methanol, temperature: 40° C., flow rate: 1.0 mL/min., and UV detection: 254 nm by use of an optical isomer-separation column (CHIRALCELOC, f4.6×250 mm).

### Example 1

### Solvent: Methanol

In 25.0 g of methanol, 1.00 g of a racemate of compound of formula (1) was dissolved at 62° C., and then the resulting solution was cooled to 53° C. over 20 minutes. The solution was seeded with 10 mg of an optically active substance of compound of formula (1) ((+) form: 100% ee), cooled to 33° C. over 45 minutes, and then stirred at 30-33° C. for 1 hour. After crystal separated out was collected through filtration, it was dried under reduced pressure at 50° C. to obtain 144.8 mg of optically active substance ((+) form) of compound of formula (1) having an optical purity of 88.37% ee as pale yellow crystal. On the other hand, the filtrate was concentrated to obtain 771.6 mg of optically active substance ((−) form) of compound of formula (1) having an optical purity of 13.33% ee as yellow foamy substance.

### Example 2

### Solvent: Ethanol

In 25.0 g of ethanol, 1.00 g of a racemate of compound of formula (1) was dissolved at 73° C., and then the resulting solution was cooled to 53° C. over 20 minutes. The solution was seeded with 10 mg of an optically active substance of compound of formula (1) ((+) form: 100% ee), cooled to 35° C. over 45 minutes, and then stirred at 34-35° C. for 1 hour. After crystal separated out was collected through filtration, it was dried under reduced pressure at 50° C. to obtain 414.3 mg of optically active substance ((+) form) of compound of formula (1) having an optical purity of 11.42% ee as pale yellow crystal. On the other hand, the filtrate was concentrated to obtain 571.4 mg of optically active substance ((−) form) of compound of formula (1) having an optical purity of 4.35% ee as yellow foamy substance.

### Example 3

### Solvent: 1-propanol

In 25.0 g of 1-propanol, 1.00 g of a racemate of compound of formula (1) was dissolved at 87° C., and then the resulting solution was cooled to 47° C. over 55 minutes. The solution was seeded with 10 mg of an optically active substance of compound of formula (1) ((+) form: 100% ee), cooled to 30° C. over 50 minutes, and then stirred at the same temperature for 1 hour. After crystal separated out was collected through filtration, it was dried under reduced pressure at 50° C. to obtain 209.9 mg of optically active substance ((+) form) of compound of formula (1) having an optical purity of 52.49% ee as pale yellow crystal. On the other hand, the filtrate was concentrated to obtain 790 mg of optically active substance ((−) form) of compound of formula (1) having an optical purity of 15.45% ee as yellow foamy substance.

### Example 4

### Solvent: Ethyl Acetate

In 30.0 g of ethyl acetate, 1.00 g of a racemate of compound of formula (1) was dissolved at 73° C., and then the resulting solution was cooled to 42° C. over 20 minutes. The solution was seeded with 10 mg of an optically active substance of compound of formula (1) ((+) form: 100% ee), cooled to 20° C. over 40 minutes, and then stirred at the same temperature for 40 minutes. After crystal separated out was collected through filtration, it was dried under reduced pressure at 50° C. to obtain 87.4 mg of optically active substance ((+) form) of compound of formula (1) having an optical purity of 93.00% ee as pale yellow crystal. On the other hand, the filtrate was concentrated to obtain 920 mg of optically active substance ((−) form) of compound of formula (1) having an optical purity of 9.14% ee as yellow foamy substance.

### Example 5

### Repeated Preferential Crystallization

In 125 g of MeOH, 5.00 g of a racemate of compound of formula (1) was dissolved at 62° C., and then the resulting solution was cooled to 51° C. over 30 minutes. The solution was seeded with 50 mg of an optically active substance of compound of formula (1) ((+) form: 100% ee), cooled to 30° C. over 80 minutes, and then stirred at the same temperature for 60 minutes. After crystal separated out was collected through filtration, it was dried under reduced pressure at 50° C. to obtain 0.568 g of optically active substance ((+) form) of compound of formula (1) having an optical purity of 89.97% ee as pale yellow crystal. In addition, optically active substance ((−) form) of compound of formula (1) having an optical purity of 12.04% ee was present in the filtrate. In the filtrate, 0.602 g of a racemate of compound of formula (1) was added, dissolved with heat and cooled to 50° C. over 50 minutes. The solution was seeded with 50 mg of an optically active substance of compound of formula (1) ((−) form: 100% ee), cooled to 30° C. over 75 minutes, and then stirred at the same temperature for 60 minutes. After crystal separated out was collected through filtration, it was dried under reduced pressure at 50° C. to obtain 1.364 g of optically active substance ((−) form) of compound of formula (1) having an optical purity of 86.92% ee as pale yellow crystal. In addition, optically active substance ((+) form) of compound of formula (1) having an optical purity of 17.87% ee was present in the filtrate. In the filtrate, 1.360 g of a racemate of compound of formula (1) and 15.0 g of methanol were added, dissolved with heat again and cooled to 54° C. over 35 minutes. The solution was seeded with 50 mg of an optically active substance of compound of formula (1) ((+) form: 100% ee), cooled to 30° C. over 55 minutes, and then stirred at the same temperature for 55 minutes. After crystal separated out was collected through filtration, it was dried under reduced pressure at 50° C. to obtain 1.270 g of optically active substance ((+) form) of compound of formula (1) having an optical purity of 93.05% ee as pale yellow crystal. In addition, optically active substance ((−) form) of compound of formula (1) having an optical purity of 14.23% ee was present in the filtrate.

### Example 6

### Purification by Recrystallization, Case 1

A mixture of 899.6 mg of an optically active substance of compound of formula (1) ((+) form: 100% ee) with 100.2 mg of a racemate of compound of formula (1) was dissolved in 25.0 g of ethanol at 77° C. An optical purity was 88.80% ee at this point. The resulting solution was cooled to 28° C. over 35 minutes, and then cooled further to 23° C. over 45 minutes. After crystal separated out was collected through filtration, it was dried under reduced pressure at 50° C. to obtain 891 mg of optically active substance ((+) form) of compound of formula (1) having an optical purity of 99.71% ee as pale yellow crystal. On the other hand, the filtrate was concentrated to obtain 0.10 g of optically active substance ((−) form) of compound of formula (1) having an optical purity of 7.42% ee as yellow foamy substance.

### Example 7

### Purification by Recrystallization, Case 2

A mixture of 80.6 mg of an optically active substance of compound of formula (1) ((+) form: 100% ee) with 420.4 mg of a racemate of compound of formula (1) was dissolved in 12.5 g of ethanol at 76° C. An optical purity was 16.70% ee at this point. The resulting solution was cooled to a temperature of 5° C. or below over 40 minutes, and stirred at the same temperature for 30 minutes. After crystal separated out was collected through filtration, it was dried under reduced pressure at 50° C. to obtain 371.7 mg of optically active substance ((+) form) of compound of formula (1) having an optical purity of 26.01% ee as yellow solid. In addition, the filtrate was concentrated to obtain 0.12 g of optically active substance ((−) form) of compound of formula (1) having an optical purity of 7.55% ee as yellow foamy substance.

### Example 8

### Purification by Toluene Crystallization

A mixture of 51.7 mg of an optically active substance of compound of formula (1) ((+) form: 100% ee) with 449.0 mg of a racemate of compound of formula (1) was dissolved in 7.50 g of toluene at 95° C. An optical purity was 10.20% ee at this point. The resulting solution was cooled to 60° C. over 7 minutes. Thereafter, the solution was seeded with 5.2 mg of toluene solvate of a racemate of compound of formula (1), cooled to a temperature of 5° C. or below over 40 minutes and stirred at the same temperature for 30 minutes. After crystal separated out was collected through filtration, it was dried under reduced pressure at 60° C. to obtain 506.8 mg of toluene solvate of a racemate of compound of formula (1) (as actually an optically active substance ((+) form) of compound of formula (1) was in slightly excess, an optical purity was 5.65% ee) as yellow solid. In addition, the filtrate was concentrated to obtain 0.05 g of optically active substance ((+) form) of compound of formula (1) having an optical purity of 97.96% ee as yellow residue.

### Example 9

### Preferential Crystallization in the Presence of Methanesulfonic Acid

In 3.00 (4.75 mmol) g of a racemate of compound of formula (1), 300 mg (3.12 mmol, 0.66 equivalent) of methanesulfonic acid was added, the resulting solution was refluxed with heat (62° C.) in 15.0 g of methanol, and dissolved. The resulting solution was cooled to 50° C. over 30 minutes, and then 10 mg of an optically active substance of compound of formula (1) ((+) form: 100% ee) was added, cooled to 40° C. over 1 hour, and then stirred at the same temperature for 1 hour. After crystal separated out was collected through filtration, it was washed with 2 ml of methanol and dried under reduced pressure at 50° C. to obtain 716.2 mg of optically active substance ((+) form) of compound of formula (1) having an optical purity of 30.62% ee as white solid. On the other hand, optically active substance ((−) form) of compound of formula (1) present in 14.42 g of the filtrate had 3.82% ee.

Industrial Applicability

The present invention establishes a process for producing an optically active efonidipine that is economically excellent.

1. A process for producing an optically active substance of compound of formula (1) 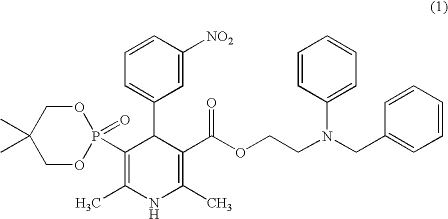
characterized by comprising:
dissolving a racemate of the compound of formula (1) in a solvent to prepare a supersaturated solution; and
adding crystal of either one optically active substance of the compound of formula (1) as a seed crystal in the supersaturated solution to allow crystal of the one optically active substance added as the seed crystal to separate out; or
dissolving a mixture of the compound of formula (1) in which either one optically active substance thereof is present in excess in a solvent to prepare a supersaturated solution; and
adding crystal of the one optically active substance present in excess as a seed crystal in the supersaturated solution to allow crystal of the one optically active substance present in excess to separate out.

2. The process for producing an optically active substance according to claim 1, wherein the solvent is alcohols or esters.

3. A process for purifying an optically active substance of compound of formula (1) 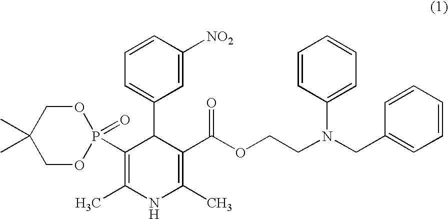
characterized by comprising:
recrystallizing a mixture of the compound of formula (1) in which either one optically active substance thereof is present in excess, or
allowing crystal to separate out from a solution of a mixture of the compound of formula (1) in which either one optically active substance thereof is present in excess, as a result of it, an optically active substance present in excess in a mother liquor from which the crystal is removed is the other optically active substance that is not one of the crystal.

4. The process for purifying an optically active substance according to claim 3, wherein the solvent is alcohols or esters.

5. A process for producing an optically active substance of compound of formula (1) 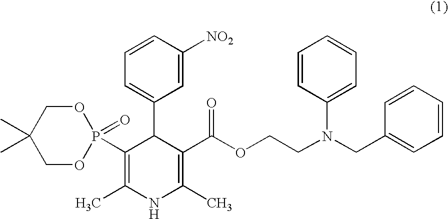
characterized by comprising:
dissolving a mixture of the compound of formula (1) in which either one optically active substance thereof is present in excess in an aromatic hydrocarbon of formula (2) 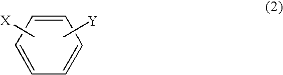
wherein X and Y are identical with or different from each other, hydrogen atom, C₁₋₃alkyl group, halogen atom or C₁₋₃alkoxy group, to prepare a supersaturated solution;
crystallizing a solvate of a racemate of the compound of formula (1) and the aromatic hydrocarbon from the supersaturated solution;
removing resulting crystal, and then obtaining in a high purity the one optically active substance present in excess of the compound of formula (1).
